(19) United States
(12) Patent Application Publication
(10) Pub. No.: US 2022/0249327 A1
(43) Pub. Date: Aug. 11, 2022

(54) BLISTER PACK CONTENT MONITORING

(71) Applicant: HealthBeacon Limited, Dublin (IE)

(72) Inventors: James JOYCE, Needham, MA (US); Kieran DALY, Dublin (IE); Richard SHATTOCK, Dublin (IE)

(21) Appl. No.: 17/622,144

(22) PCT Filed: Jun. 26, 2019
(86) PCT No.: PCT/US2019/039275

§ 371 Date: Dec. 22, 2021

(51) Int. Cl. A61J 7/04; A61J 1/03

(52) U.S. Cl. CPC A61J 7/0436; A61J 1/035; A61J 2200/72; A61J 2200/30; A61J 2200/70; A61J 7/0481

(57) ABSTRACT

A cartridge includes a case housing a smartphone, a housing configured to hold a medication blister pack, and a detection method to record the blister pack being put in and out of the housing.

### BACKGROUND OF THE INVENTION

The present invention relates generally to blister packs, and more particularly blister pack content monitoring.

Medications, including prescription and over-the-counter pharmaceuticals, as well as vitamins and other dietary supplements, form a mainstay of health care, maintenance, and disease management and prevention. Typically a medication is given in repeated oral doses, usually as pills (here taken to include capsules), spread out over time so as to sustain desired levels of active ingredients in the patient's body. Any substantial deviation from the recommended timing, such as missing a dose or “doubling up” on doses, may decrease a medication's effectiveness or cause outright harm to the patient.

In general, a blister pack is a transparent, molded piece of plastic, often sealed to a sheet of cardboard, used to package small items such as tablets. With substantial and increasing medication use comes increased risk in medication errors, compounded by an increase in the aging population with increasingly complex medical needs and introduction of new medications. It is not uncommon for patients, particularly elderly, to encounter problems removing pills or tablets from the packaging, sometimes dropping or losing them in the process. In addition, elderly patients often report problems with tablets breaking or crumbling when removing them from the blister pack and often reported confusion in identifying medicines that have similar packaging.

Moreover, some reports have shown that one in five patients over the age of 50 take five or more medications with the median in over 50's being two medications. Polypharmacy can increase the risk of non-adherence, in appropriate prescribing or increased adverse events.

What is needed is a system and method to ensure that medications are being taken on the prescribed schedule.

### BRIEF SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

In general, in one aspect, the invention features a cartridge including a case housing a smartphone, a housing configured to hold a medication blister pack, and a detection method to record the blister pack being put in and out of the housing.

Embodiments may have one or more of the following advantages.

The present invention provides a solution to manage single or multiple digital platforms through a medication management system.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

These and other features, aspects, and advantages of the present invention will become better understood with reference to the following description, appended claims, and accompanying drawings where:

FIG. 1 illustrates a first embodiment of an exemplary blister pack device in accordance with the present invention.

FIG. 2 illustrates an exemplary blister pack being slid into a compartment.

FIG. 3A illustrates a lever mechanism being lifted from a home position.

FIG. 3B illustrates the lever being pushed to release a pill from a blister pack.

FIG. 3C illustrates the lever mechanism in a closed or home position.

FIG. 4 illustrates an exemplary tablet dispensing mechanism.

FIG. 5 illustrates another exemplary blister pack system.

FIG. 6 illustrates an exemplary user interface (UI).

### DETAILED DESCRIPTION OF THE INVENTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

The present invention relates to a packaging device and use-monitoring system for items intended to be dispensed over a period of time or on a particular schedule, such as prescription medications. The present invention can be directed towards patients who would benefit from easier access to their medication. The medicine management system may be connected to external networks and/or system and thus serve as an adherence management system to assist with tracking a patient's medication schedule and adherence to such schedule.

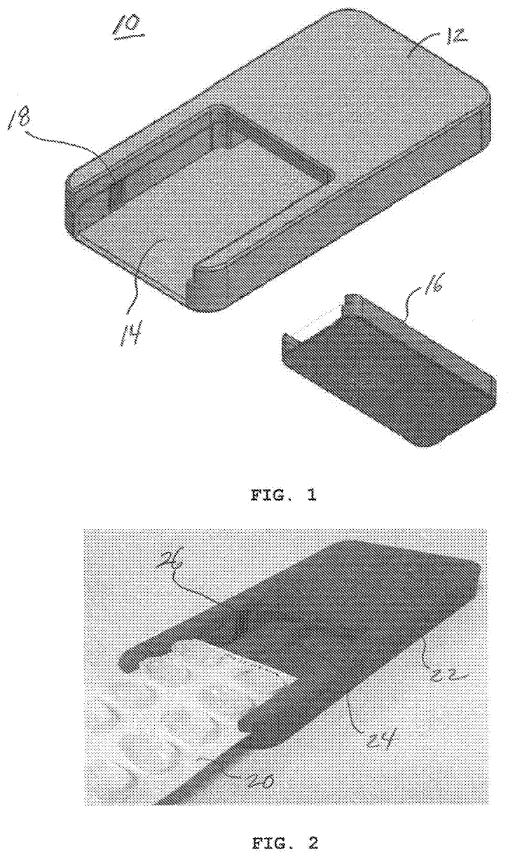

In FIG. 1, a first embodiment of an exemplary blister pack device 10 includes a container 12 that houses a medication blister pack (not shown) within an enclosed compartment 14. In the embodiment shown, the enclosed compartment 14 is accessed using a door or lid 16. In one particular implementation, the container 12 may be incorporated into or be part of a smartphone case (not shown).

The door or lid 16 can include a sensor 18 that is configured to register an event, such as an opening and/or closing of the door or lid 16 and removal of the blister pack. The event may then be recorded in a smartphone by a smartphone adherence app (fully described below) and/or transmitted wirelessly by the smartphone to a remote system (not shown). Once received by the remote system, the event may be recorded against an owner's profile, such as a patient profile. Historical information can be accumulated over time and used, for example, to reconcile the owner's prescription schedule. When the owner opens the door or lid 16 and accesses the blister pack a pill is dispensed as normal. Once the blister pack is returned to the compartment the door or lid 16 is closed. The closure of the door or lid 16 is a recordable event and saved by the adherence smartphone app and/or remote system.

In FIG. 2, an exemplary blister pack 20 is shown illustrated being slid into a compartment 22 and held in place by a lip, clip or clasp 24, which, in specific implementation, may be released by a button or lever. Once released and the blister pack is slid out, a sensor 26 within the compartment 22 and/or button can register an event.

In one embodiment, a medication management system can be programmed with a patient's prescription schedule that can include, for example, a combination of pills, tablets, injectable or liquid medications. In one example, the medication management system can signal to a patient (via a blistie smartphone app, fully described below) that they are due to take an injection which when administered is disposed of into a connected sharps container. This is recorded as a positive digital event. The system then signals through the blistie smartphone app that the patient is due to take their first co-prescribed tablet. The patient accesses their blister pack by sliding it out of the compartment, this action is taken as an indication that they accessed their medication at the appropriate time. The event is reconciled back to digital management system. The system therefore has the ability to manage multiple digital devices and single or multiple medication tracking.

In one implementation, when the blister pack device 10 (in FIG. 1) is associated with a smartphone, the smartphone's accelerometer can be used to register an event and even capture a unique digital signature.

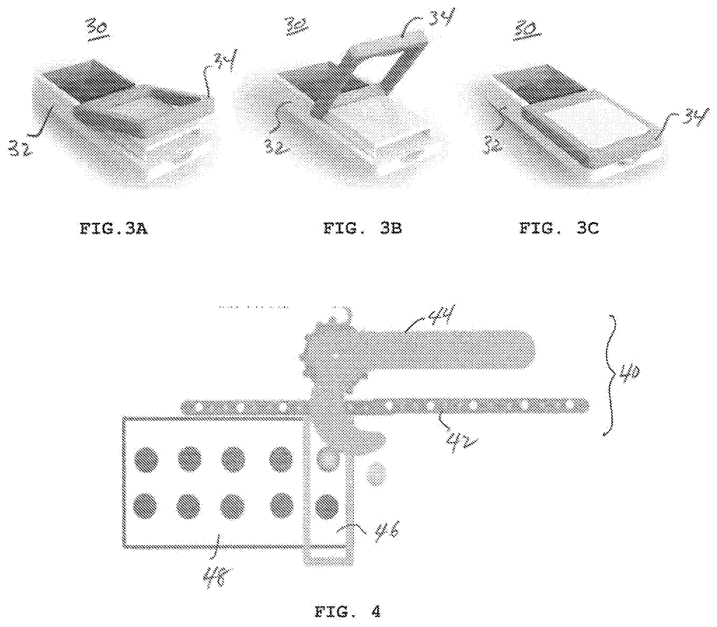

In FIG. 3A, another embodiment of an exemplary blister pack device 30 includes a container 32 that houses a blister pack (not shown). The container 32 includes a tablet dispensing mechanism 34 that is activated by a button or lever. The tablet dispensing mechanism 34 reduces a force required to dispense a tablet and maintains an integrity of the tablet, reducing the chances of crumbling or damage. One such tablet dispensing mechanism is a lever. As seen in FIG. 3A, a user can lift the lever to load a tablet in a blister pack into position. In FIG. 3B, pushing the lever releases the tablet for user consumption. A sensor (not shown) in the container 32 and/or lever 34 can record an event of utilizing the lever. In FIG. 3C, the lever is returned to its home position and the user retrieves a pill.

In FIG. 4, another tablet dispensing mechanism is a ratchet system 40. The ratchet system 40 pulls pills along a track 42 into a “punching” position as a lever 44 is lifted. As the lever 44 comes back down, pills at a window 46 are forced out of the blister pack 48 into a channel. The film that makes up the blister pack 48 does not get stuck as the lever 44 is lifted to load the next round of pills, while an empty bluster pack 48 is slid inside the compartment (not shown).

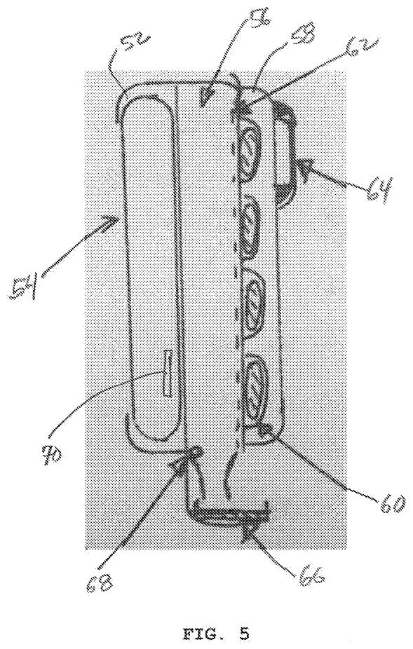

In FIG. 5, another exemplary blister pack system 50 includes a case 52 that houses a smartphone 54, a cavity 56 and a housing 58 to hold a medication blister pack 60. A foil management membrane 62 provides a barrier between the blister pack 60 and the cavity 56 and facilitates a release of a tablet or pill from foil that makes up the blister pack 60.

An outer portion of the housing 58 includes a release device 64, such as a push button. The button 64 reduces a force required to push a tablet through the sealed membrane of the blister pack 60 and records a digital event each time it is depressed.

More specifically, a tablet/pill is guided to a dispensing tray 66 which can be easily presented to a patient. A sensor/camera 68 resides in a bottom of the cavity 56 and records an event and/or image of the dispensed pill. The event and/or image may stored in the smartphone 54 or transmitted wirelessly to a server in a remote network of interconnected computers (not shown). Information stored on the server in remote network can be used to manage patient adherence data.

As mentioned previously, the smartphone 54 may include a blistie smartphone app 70 residing in the smartphone's memory, also referred to as the patient companion app. The blistie smartphone app 70 provides a user interface (UI) to assist with a management of the patient's schedule. The blistie smartphone app 70 adds another level of user experience and value to patients. The blistie smartphone app 70 allows patients to have more control over their treatment, enabling them to customize their reminders, report when medication is taken when away from the blister pack, gain insights into their treatment and share this data with a caretaker or export it for their healthcare provider.

For example, the blistie smartphone app 70 can remind users when and where to take medication, report attacks, provide a treatment history, store nurse case manager contact details, provide, treatment education, and so forth. The blistie smartphone app 70 can be used to mark injections as taken along with a photograph in the injection site, report drops away from the device, enable the user to view their data and export their treatment history, calendar events and contact help/assistance.

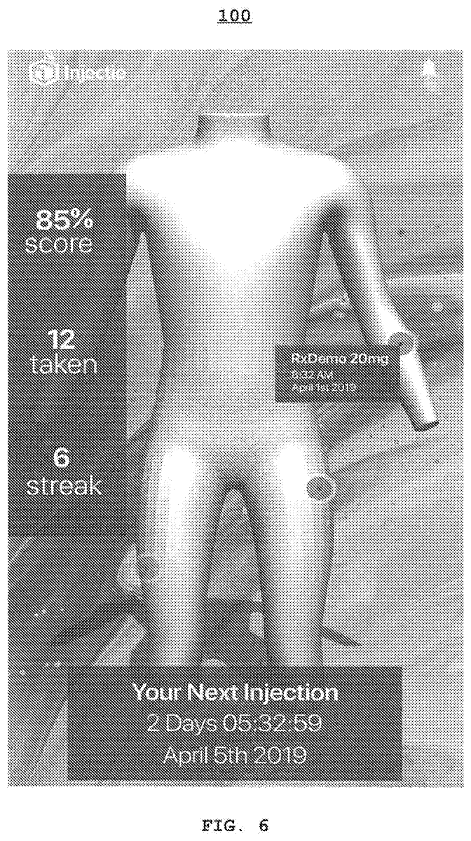

The blistie smartphone app 70 does these and other functions with a series of easy to use displays, i.e., specific user-friendly user interfaces. In FIG. 6, an exemplary user interface (UI) 100 generated by the blistie smartphone app 70 is shown. The UI 100 illustrates various pertinent information pertaining to a patient's reoccurring schedule of injections.

It would be appreciated by those skilled in the art that various changes and modifications can be made to the illustrated embodiments without departing from the spirit of the present invention. All such modifications and changes are intended to be within the scope of the present invention except as limited by the scope of the appended claims.

What is claimed:

1. A cartridge comprising:
a case housing a smartphone;
a housing configured to hold a medication blister pack; and
a detection method to record the blister pack being put in and out of the housing.

2. The cartridge of claim 1 further comprising a lever or push button that attaches to a dispensing mechanism.

3. The cartridge of claim 2 wherein the dispensing mechanism is configured to reduce a force required to push the tablet out of the blister pack and maintain an integrity of the tablet.

4. The cartridge of claim 3 wherein the dispensing mechanism is further configured to:
incrementally feed the blister pack through the cartridge;
register a digital event each time the lever or button is operated; and
register a tablet drop event.

5. The cartridge of claim 1 further comprising a protective heat resistant film between the case and the smartphone.

6. The cartridge of claim 1 wherein an accelerometer in the smartphone provides a digital signature of a dispensed tablet.

7. The cartridge of claim 1 further comprising a cavity positioned between the phone and the blister pack.

8. The cartridge of claim 7 wherein the cavity includes a sensor/camera at a bottom for recording an image/event of a dispensed tablet.

9. The cartridge of claim 8 wherein the sensor/camera is configured to recognize when the cavity is empty.

10. The cartridge of claim 1 wherein the smartphone comprising an app is configured to assist in managing and tracking the patient's medication schedule to train and help develop strong adherence habits.

11. The cartridge of claim 10 wherein the app is further configured to wirelessly communicate with a medication management system residing on a remote server.

12. The cartridge of claim 11 wherein the medication management system enables a patient support provider to assist patients with their medication schedules, determine if they are struggling with their routine and intervene as appropriate.

13. The cartridge of claim 12 wherein the medication management system enables reconciliation of prescription schedule with tablet drop schedule.

14. The cartridge of claim 7 wherein the app is further configured to monitor and control the temperature of the cavity.
